(19) United States
(12) Patent Application Publication
(10) Pub. No.: US 2024/0111837 A1
(43) Pub. Date: Apr. 4, 2024

(54) MODULE FOR IDENTIFICATION AND CLASSIFICATION TO SORT CELLS BASED ON THE NUCLEAR TRANSLOCATION OF FLUORESCENCE SIGNALS

(71) Applicants: SONY GROUP CORPORATION, Tokyo (JP); Sony Corporation of America, New York, NY (US)

(72) Inventors: Ming-Chang Liu, San Jose, CA (US); Su-Hui Chiang, San Jose, CA (US); Haipeng Tang, Sunnyvale, CA (US); Michael Zordan, Boulder Creek, CA (US); Ko-Kai Albert Huang, Cupertino, CA (US)

(21) Appl. No.: 18/113,753

(22) Filed: Feb. 24, 2023

Related U.S. Application Data
(60) Provisional application No. 63/377,788, filed on Sep. 30, 2022.

(51) Int. Cl. G06F 18/2415; G06N 3/091; G16B 20/00

(52) U.S. Cl. CPC G06F 18/2415; G06N 3/091; G16B 20/00

(57) ABSTRACT

An Image Activated Cell Sorting (IACS) classification workflow includes: employing a neural network-based feature encoder (or extractor) to extract features of cell images; automatically clustering cells based on extracted cell features; identifying a cluster to pick which cluster(s) to sort based on the cell images; fine-tuning a classification network based on the cluster(s) selected; and once refined, the classification network is used to sort cells for real-time live sorting.

### CROSS-REFERENCE TO RELATED APPLICATION(S)

This application claims priority under 35 U.S.C. § 119(e) of the U.S. Provisional Patent Application Ser. No. 63/377,788, filed Sep. 30, 2022 and titled, “MODULE FOR IDENTIFICATION AND CLASSIFICATION TO SORT CELLS BASED ON THE NUCLEAR TRANSLOCATION OF FLUORESCENCE SIGNALS,” which is hereby incorporated by reference in its entirety for all purposes.

### FIELD OF THE INVENTION

The present invention relates to cell sorting. More specifically, the present invention relates to image-based cell sorting.

### BACKGROUND OF THE INVENTION

Traditional fluorescence activated cell sorting relies on labeling cells with fluorescent markers and has very limited morphological information of cells. However, some applications require morphological information of cells to accurately sort the cells, while some applications are not suitable to use fluorescent markers. In addition, traditional fluorescence activated cell sorting (FACS) uses manual gating to establish sorting criteria based on fluorescent markers. However, manual gating is time consuming and may be biased.

Some studies proposed image based cell sorting using supervised learning based on deep neural networks or hand crafted features. They assumed cell images with ground truth for training, which may not be available. Some software that helps the gating process rely on particular hand-crafted features of fluorescent markers, which may not have sufficient morphological information for some applications or may not be suitable for some other applications.

### SUMMARY OF THE INVENTION

An Image Activated Cell Sorting (IACS) classification workflow includes: employing a neural network-based feature encoder (or extractor) to extract features of cell images; automatically clustering cells based on extracted cell features; identifying a cluster to pick which cluster(s) to sort based on the cell images; fine-tuning a classification network based on the cluster(s) selected; and once refined, the classification network is used to sort cells for real-time live sorting.

In one aspect, a method comprises extracting one or more features from cell images using a neural network-based feature encoder, clustering one or more cells from the cell images based on the extracted one or more features to generate one or more clusters, identifying a cluster of the one or more clusters to sort, fine-tuning a classification network based on the cluster and performing real-time live sorting of a set of cells using the classification network. The one or more features comprise a target protein based on a fluorescent dye. Clustering the one or more cells is based on a location of the target protein. When the target protein is in the cytosol, the one or more cells are clustered as dormant cells, and when the target protein is in the nucleus, the one or more cells are clustered as activated cells. Identifying the cluster to sort is based on a user manually identifying the cluster. Identifying the cluster to sort is based on machine learning to identify the cluster. Wherein fine-tuning the classification network includes performing training with an additional dataset based on the cluster.

In another aspect, an apparatus comprises a non-transitory memory for storing an application, the application for: extracting one or more features from cell images using a neural network-based feature encoder, clustering one or more cells from the cell images based on the extracted one or more features to generate one or more clusters, identifying a cluster of the one or more clusters to sort, fine-tuning a classification network based on the cluster and performing real-time live sorting of a set of cells using the classification network and a processor configured for processing the application. The one or more features comprise a target protein based on a fluorescent dye. Clustering the one or more cells is based on a location of the target protein. When the target protein is in the cytosol, the one or more cells are clustered as dormant cells, and when the target protein is in the nucleus, the one or more cells are clustered as activated cells. Identifying the cluster to sort is based on a user manually identifying the cluster. Identifying the cluster to sort is based on machine learning to identify the cluster. Fine-tuning the classification network includes performing training with an additional dataset based on the cluster.

In another aspect, a system comprises a first computing device configured for sending one or more cell images to the second computing device and a second computing device configured for: extracting one or more features from the one or more cell images using a neural network-based feature encoder, clustering one or more cells from the cell images based on the extracted one or more features to generate one or more clusters, identifying a cluster of the one or more clusters to sort, fine-tuning a classification network based on the cluster and performing real-time live sorting of a set of cells using the classification network. The one or more features comprise a target protein based on a fluorescent dye. Clustering the one or more cells is based on a location of the target protein. When the target protein is in the cytosol, the one or more cells are clustered as dormant cells, and when the target protein is in the nucleus, the one or more cells are clustered as activated cells. Identifying the cluster to sort is based on a user manually identifying the cluster. Identifying the cluster to sort is based on machine learning to identify the cluster. Fine-tuning the classification network includes performing training with an additional dataset based on the cluster.

### BRIEF DESCRIPTION OF THE DRAWINGS

FIG. 1 illustrates a diagram of nuclear translocation of proteins during cell activation according to some embodiments.

FIG. 2 illustrates a flowchart of Image Activated Cell Sorting (IACS) classification workflow according to some embodiments.

FIG. 3 illustrates a diagram of an integrated nuclear translocation module according to some embodiments.

FIG. 4 illustrates a diagram of an integrated nuclear translocation module according to some embodiments.

FIG. 5 illustrates a diagram of results of an exemplary implementation according to some embodiments.

FIG. 6 shows a block diagram of an exemplary computing device configured to implement the identification and classification implementation according to some embodiments.

### DETAILED DESCRIPTION OF THE PREFERRED EMBODIMENT

An identification and classification implementation is used to sort cells based on the nuclear translocation of fluorescence signals. The identification and classification implementation is able to utilize the clustering implementation as described in U.S. patent application Ser. No. 18/070,352, filed Nov. 28, 2022, titled, “IMAGE-BASED UNSUPERVISED MULTI-MODEL CELL CLUSTERING,” which is hereby incorporated by reference in its entirety for all purposes.

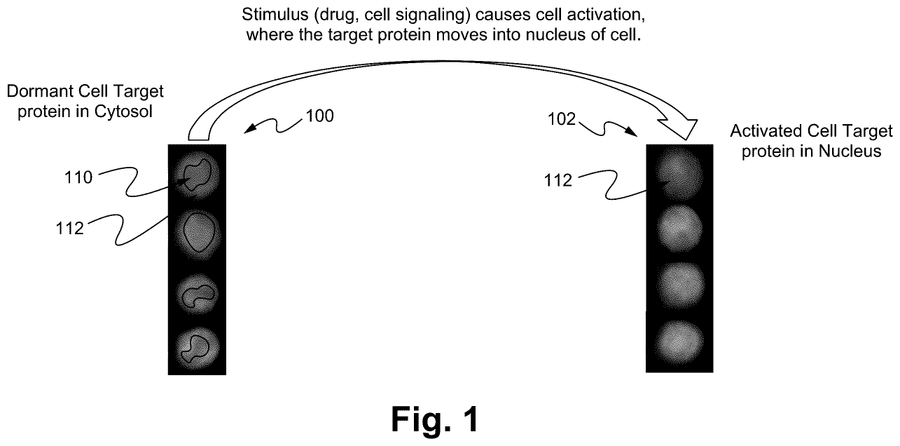

FIG. 1 illustrates a diagram of nuclear translocation of proteins during cell activation according to some embodiments. In the Figure, in the dormant cells 100, a nucleus 110 is labeled with a red fluorescent dye, and a target protein 112 is labeled with a green fluorescent dye. An outline is provided in the Figure to help distinguish between the nucleus 110 and the protein 112, although there may not be a distinct line of separation in the real world. After activation, the activated cells 102 have the target protein 112 throughout the cell, including the nucleus, so the entire cell appears as one color (e.g., green) or mostly one color.

A protein, typically in the cytosol/outside of the nucleus of a cell, is labeled with fluorescence. When a cell is activated (e.g., by a disease, drug or other external stimulus), the protein moves into the nucleus to promote gene expression and protein expression. For example, a nucleus is labeled with a red fluorescent dye, and a protein (or multiple proteins) is labeled with a green fluorescent dye. The target proteins are typically from the NF-Kb family, although any other target protein is able to be used. The amount of fluorescent signal does not change; rather, the location of the fluorescence changes, which is why imaging is important instead of traditional flow cytometry which merely measures the total fluorescent signal. A dormant cell (also referred to as cytosolic) is where the green fluorescence signal (or other color) is outside of the nucleus and is typically very distinct from the nucleus (e.g., red/green contrast). In an activated cell, the green fluorescence signal is able to be seen spread throughout the entire cell, including the nucleus. The total fluorescence signal does not change between a dormant cell and an activated cell, but the appearance of the cell is clearly changed. Any type of image processing or analysis is able to be used to detect the change and/or movement of color (e.g., by detecting a specific shape or a change of a shape, detecting movement of a color, detecting loss of a color, detecting an increase of amount of one color and a decrease of an amount of another color). Machine Learning (ML) and/or Artificial Intelligence (AI) are able to be used to perform the image analysis/processing. The process is able to be a permanent process (e.g., the appearance change remains) or a transient process (e.g., the appearance returns to the original state after a temporary change). For example, a user may be looking at a response to a stimulus, but the activated cell may return to a dormant cell after a period of time (e.g., 1 hour).

Nuclear translation assay is not possible on traditional cell sorters. However, with cell imaging, and cell sorting based on imaging, nuclear translocation assay is possible. Nuclear translocation assay is able to be used to sort cell images based on whether the cell stays dormant or becomes activated, and then do further studies on those cells (e.g., single-cell genomics). For example, further analysis is able to be performed to determine what causes certain cells to be activated or not. There are many pharmaceutical use cases (e.g., in the development of new medications).

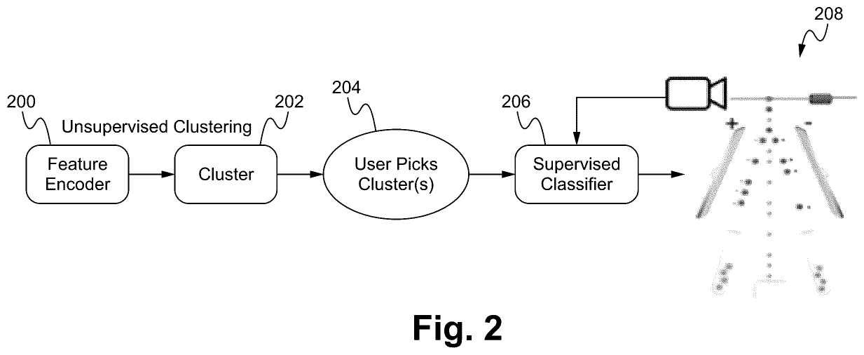

FIG. 2 illustrates a flowchart of Image Activated Cell Sorting (IACS) classification workflow according to some embodiments. In the step 200, a neural network-based feature encoder (or extractor) is employed to extract features of cell images (e.g., fluorescent dye in a target protein which may be in the cytosol or nucleus of a cell). For example, the neural network-based feature encoder is able to be trained to detect a location of a specific colored dye, an amount of the specific colored dye, a proportion of one dye versus another dye, the location of one dye versus another dye, the shape of a specific colored dye, and/or any other training for the detection of one or more features. Furthering the example, a neural network-based feature encoder is trained to detect green fluorescent dye in a target protein and red fluorescent dye in a nucleus. In some embodiments, the feature encoder is based on a multi-layer neural network. In some embodiments, the feature encoder uses several convolutional layers, followed by pooling layers. To train a feature encoder, an exemplary approach is to use contrastive loss which includes contrasting each sample to a set of positive and negative samples for computing loss. Additional datasets may be used to further refine the feature encoder after the feature encoder has been trained.

In the step 202, cells are automatically clustered based on extracted cell features. For example, a neural network-based feature encoder is trained to detect green fluorescent dye in a target protein and red fluorescent dye in a nucleus, and when the green dye is on the outer part of a cell with a red center, the cell is able to be classified/clustered as dormant, whereas if the green dye is dispersed throughout the cell with little to no red dye remaining, the cell is classified/clustered as activated. Clustering separates and groups different types of cells based on the extracted features (e.g., target protein in the cytosol, target protein in a nucleus, or ambiguity of where the target protein is). In some embodiments, the clustering is optional. In some embodiments, the clustering optionally provides feedback for training the feature extractor. Clustering may utilize hierarchical density-based clustering or other clustering algorithms. Hierarchical Density-Based Spatial Clustering (HDBSCAN) is an exemplary clustering algorithm that is able to handle an unknown number of classes. HDBSCAN performs density-based clustering with noise over epsilon values and integrates the result to find stable clustering. Given a set of points in some space, HDBSCAN groups together points that are closely packed together (e.g., a point with many nearby neighbors). Although HDBSCAN is described herein, any clustering algorithm is able to be utilized.

In the step 204, the user is then able to look at the cell images to identify the cluster to pick which cluster(s) or type of cluster(s) to sort. For example, a user may want to focus on cells that are activated to perform further analysis as to why those cells became activated (e.g., what do those cells have in common with each other to cause activation). In some embodiments, the determination of the types of cells in the cluster is able to be automated using ML/AI or another matching/identifying implementation. Similarly, ML/AI is able to be used to select which clusters to sort. For example, if a drug is being tested, and the AI knows (from previous learning) that the goal is to figure out why the drug is activated in certain cells, the AI is able to automatically select the correct cluster for further sorting.

In the step 206, the classification network (e.g., neural network using AI) is fine-tuned based on cluster(s) selected by the user or ML/AI. Fine-tuning is able to be implemented in any manner such as performing additional ML. For example, one or more additional datasets are used to train the classification network. The additional datasets are able to be related to the selected clusters (e.g., if the cluster is activated cells, then the classification network receives additional datasets of activated cells for training or other fine-tuning).

In the step 208, once refined, the classification network (e.g., neural network using AI) is used to sort cells for real-time live sorting. In some embodiments, cell sorting involves taking cells from an organism and separating them according to their type. In image-based cell sorting, the cells are able to be separated based on extracted features of cell images (e.g., the location of a target protein and/or an amount of nucleus that is visible). The real-time sorting is able to utilize the definitions of the clusters. For example, the system compares features/components of a cell and determines which cluster the cell matches most closely.

In some embodiments, the order of the steps is modified. In some embodiments, fewer or additional steps are implemented. For example, if a user is performing a nuclear translocation assay, then the clustering and the supervised classifier have pre-trained feature extractors to be used instead of a general use case version of the workflow. The IACS classification workflow is further described in U.S. patent application Ser. No. 18/070,352, filed Nov. 28, 2022, titled, “IMAGE-BASED UNSUPERVISED MULTI-MODEL CELL CLUSTERING.”

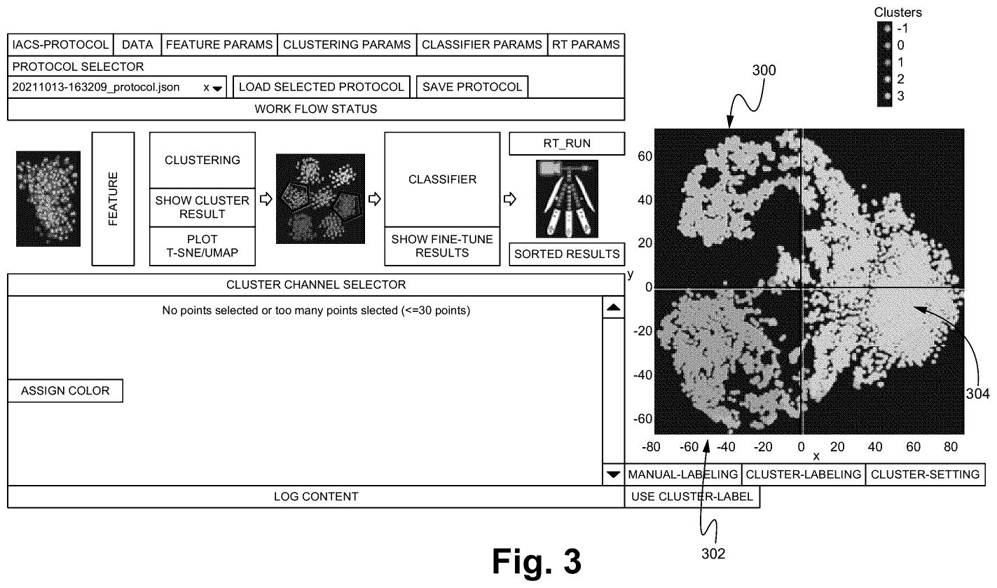

FIG. 3 illustrates a diagram of an integrated nuclear translocation module according to some embodiments. A portion of a sample (e.g., 10,000 to 100,000 cells) is run to perform unsupervised clustering. The unsupervised clustering groups cells with similar image features and plots the events on a visualization (t-SNE, UMAP) where the clusters are color-coded. For example, five clusters were found, although there are three main clusters (300, 302 and 304). Furthering the example, cluster 300 includes cells with a nuclear signal, cluster 302 includes cells with a cytosolic signal and cluster 304 includes cells that are ambiguous (e.g., not clearly nuclear or cytosolic).

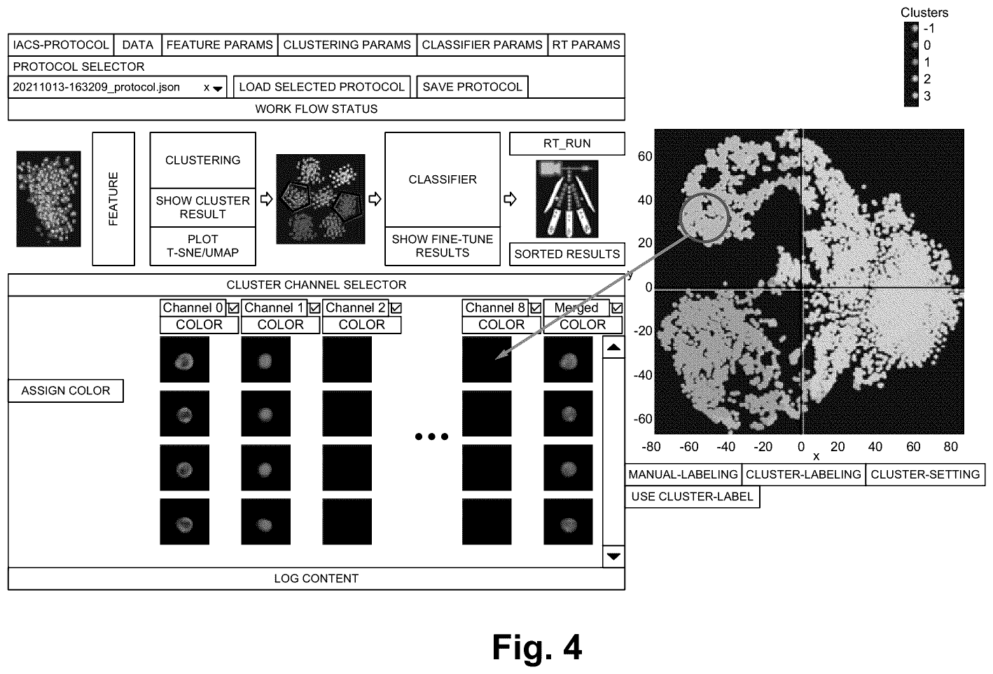

FIG. 4 illustrates a diagram of an integrated nuclear translocation module according to some embodiments. A user is able to pick a cluster and look at example cells. By looking at the example cells, the user is able to determine what cells the cluster includes (e.g., cytosolic, nuclear or other). In some embodiments, the determination of the types of cells in the cluster is able to be automated using ML and/or AI or another matching/identifying implementation.

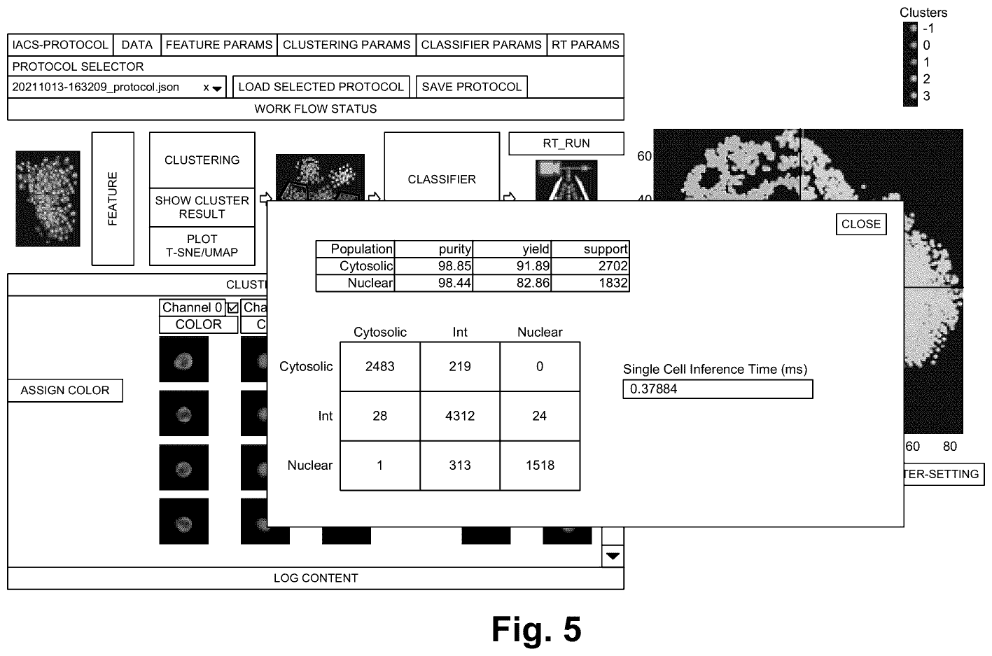

In some embodiments, after a cluster is selected by a user, a supervised classifier is refined (which takes 30 seconds to 1 minute) based on the cluster selection. Then, the supervised classifier is used to make sort decisions in real-time. In an exemplary implementation with results shown in FIG. 5, nuclear and cytosolic cells were classified with greater than 98.4% precision and greater than 80% recall, with a per cell classification time under 0.4 ms.

The identification and classification implementation is able to be performed using a GPU-based neural network, an Application-Specific Integrated Circuit (ASIC), an Field-Programmable Gate Array (FPGA), an AI-based convolutional neural network, or any other implementation.

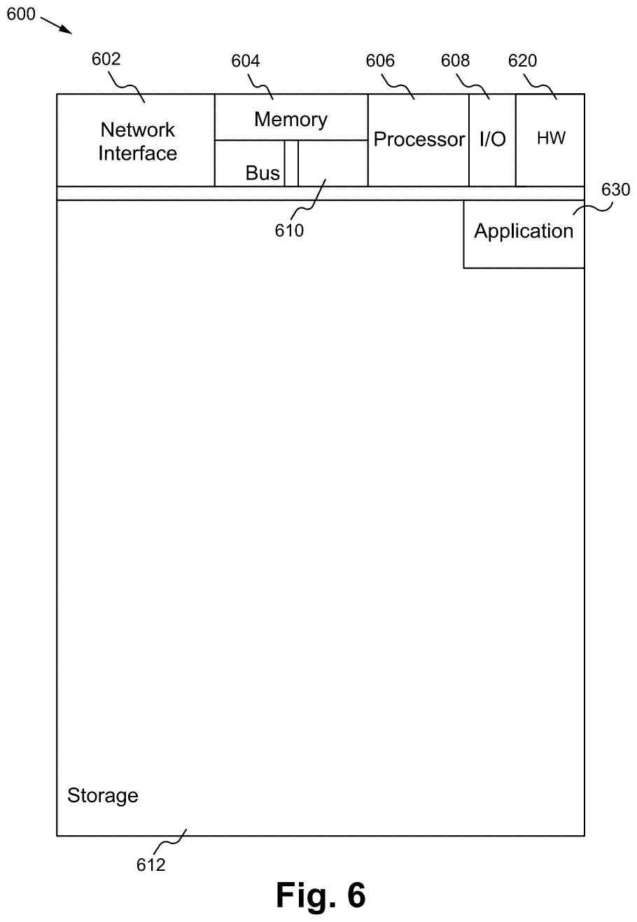

FIG. 6 shows a block diagram of an exemplary computing device configured to implement the identification and classification implementation according to some embodiments. The computing device 600 is able to be used to acquire, store, compute, process, communicate and/or display information such as images and videos. The computing device 600 is able to implement any of the identification and classification aspects. In general, a hardware structure suitable for implementing the computing device 600 includes a network interface 602, a memory 604, processors 606, I/O device(s) 608, a bus 610 and a storage device 612. The choice of processor(s) is not critical as long as suitable processor(s) with sufficient speed are chosen. The processors 606 are able to include multiple Central Processing Units (CPUs). The processors 606 and/or hardware 620 are able to include one or more Graphics Processing Units (GPUs) for efficient feature extraction based on the neural network. Each GPU should be equipped with sufficient GPU memory to perform feature extraction. The memory 604 is able to be any conventional computer memory known in the art. The storage device 612 is able to include a hard drive, CDROM, CDRW, DVD, DVDRW, High Definition disc/drive, ultra-HD drive, flash memory card or any other storage device. The computing device 600 is able to include one or more network interfaces 602. An example of a network interface includes a network card connected to an Ethernet or other type of LAN. The I/O device(s) 608 are able to include one or more of the following: keyboard, mouse, monitor, screen, printer, modem, touchscreen, button interface and other devices. Identification and classification application(s) 630 used to implement the framework are likely to be stored in the storage device 612 and memory 604 and processed as applications are typically processed. More or fewer components shown in FIG. 6 are able to be included in the computing device 600. In some embodiments, identification and classification hardware 620 is included. Although the computing device 600 in FIG. 6 includes applications 630 and hardware 620 for the identification and classification implementation, the identification and classification implementation is able to be implemented on a computing device in hardware, firmware, software or any combination thereof. For example, in some embodiments, the identification and classification applications 630 are programmed in a memory and executed using a processor. In another example, in some embodiments, the identification and classification hardware 620 is programmed hardware logic including gates specifically designed to implement the identification and classification implementation.

In some embodiments, the identification and classification application(s) 630 include several applications and/or modules. In some embodiments, modules include one or more sub-modules as well. In some embodiments, fewer or additional modules are able to be included.

Examples of suitable computing devices include a personal computer, a laptop computer, a computer workstation, a server, a mainframe computer, a handheld computer, a personal digital assistant, a cellular/mobile telephone, a smart appliance, a gaming console, a digital camera, a digital camcorder, a camera phone, a smart phone, a portable music player, a tablet computer, a mobile device, a video player, a video disc writer/player (e.g., DVD writer/player, high definition disc writer/player, ultra high definition disc writer/player), a television, a home entertainment system, an augmented reality device, a virtual reality device, smart jewelry (e.g., smart watch), a vehicle (e.g., a self-driving vehicle) or any other suitable computing device.

To utilize the identification and classification implementation described herein, devices such as a flow cytometer with an imaging system (e.g., one or several cameras or detectors) are used to acquire content, and a device is able to process the acquired content. Some imaging systems do not use cameras and reconstruct images from pulse processing from photodiodes, photomultiplier tubes or other implementations. The identification and classification implementation is able to be implemented with user assistance or automatically without user involvement.

In operation, compared to other implementations, the identification and classification implementation described herein is much more precise and is faster. For example, the identification and classification implementation described herein has precision greater than 98.4% compared to an implementation based on the Pearson's Correlation Coefficient which has a precision of around 90%.

### Some Embodiments of Module for Identification and Classification to Sort Cells Based on the Nuclear Translocation of Fluorescence Signals

1. A method comprising:
   extracting one or more features from cell images using a neural network-based feature encoder;
   clustering one or more cells from the cell images based on the extracted one or more features to generate one or more clusters;
   identifying a cluster of the one or more clusters to sort;
   fine-tuning a classification network based on the cluster; and
   performing real-time live sorting of a set of cells using the classification network.
2. The method of clause 1 wherein the one or more features comprise a target protein based on a fluorescent dye.
3. The method of clause 2 wherein clustering the one or more cells is based on a location of the target protein.
4. The method of clause 3 wherein when the target protein is in the cytosol, the one or more cells are clustered as dormant cells, and when the target protein is in the nucleus, the one or more cells are clustered as activated cells.
5. The method of clause 1 wherein identifying the cluster to sort is based on a user manually identifying the cluster.
6. The method of clause 1 wherein identifying the cluster to sort is based on machine learning to identify the cluster.
7. The method of clause 1 wherein fine-tuning the classification network includes performing training with an additional dataset based on the cluster.
8. An apparatus comprising:
   a non-transitory memory for storing an application, the application for:
      extracting one or more features from cell images using a neural network-based feature encoder;
      clustering one or more cells from the cell images based on the extracted one or more features to generate one or more clusters;
      identifying a cluster of the one or more clusters to sort;
      fine-tuning a classification network based on the cluster; and
      performing real-time live sorting of a set of cells using the classification network;
and
   a processor configured for processing the application.
9. The apparatus of clause 8 wherein the one or more features comprise a target protein based on a fluorescent dye.
10. The apparatus of clause 9 wherein clustering the one or more cells is based on a location of the target protein.
11. The apparatus of clause 10 wherein when the target protein is in the cytosol, the one or more cells are clustered as dormant cells, and when the target protein is in the nucleus, the one or more cells are clustered as activated cells.
12. The apparatus of clause 8 wherein identifying the cluster to sort is based on a user manually identifying the cluster.
13. The apparatus of clause 8 wherein identifying the cluster to sort is based on machine learning to identify the cluster.
14. The apparatus of clause 8 wherein fine-tuning the classification network includes performing training with an additional dataset based on the cluster.
15. A system comprising:
   a first computing device configured for sending one or more cell images to the second computing device; and
   a second computing device configured for:
      extracting one or more features from the one or more cell images using a neural network-based feature encoder;
      clustering one or more cells from the cell images based on the extracted one or more features to generate one or more clusters;
      identifying a cluster of the one or more clusters to sort;
      fine-tuning a classification network based on the cluster; and
      performing real-time live sorting of a set of cells using the classification network.
16. The system of clause 15 wherein the one or more features comprise a target protein based on a fluorescent dye.
17. The system of clause 16 wherein clustering the one or more cells is based on a location of the target protein.
18. The system of clause 17 wherein when the target protein is in the cytosol, the one or more cells are clustered as dormant cells, and when the target protein is in the nucleus, the one or more cells are clustered as activated cells.
19. The system of clause 15 wherein identifying the cluster to sort is based on a user manually identifying the cluster.
20. The system of clause 15 wherein identifying the cluster to sort is based on machine learning to identify the cluster.
21. The system of clause 15 wherein fine-tuning the classification network includes performing training with an additional dataset based on the cluster.

The present invention has been described in terms of specific embodiments incorporating details to facilitate the understanding of principles of construction and operation of the invention. Such reference herein to specific embodiments and details thereof is not intended to limit the scope of the claims appended hereto. It will be readily apparent to one skilled in the art that other various modifications may be made in the embodiment chosen for illustration without departing from the spirit and scope of the invention as defined by the claims.

What is claimed is:

1. A method comprising:
extracting one or more features from cell images using a neural network-based feature encoder;
clustering one or more cells from the cell images based on the extracted one or more features to generate one or more clusters;
identifying a cluster of the one or more clusters to sort;
fine-tuning a classification network based on the cluster; and
performing real-time live sorting of a set of cells using the classification network.

2. The method of claim 1 wherein the one or more features comprise a target protein based on a fluorescent dye.

3. The method of claim 2 wherein clustering the one or more cells is based on a location of the target protein.

4. The method of claim 3 wherein when the target protein is in the cytosol, the one or more cells are clustered as dormant cells, and when the target protein is in the nucleus, the one or more cells are clustered as activated cells.

5. The method of claim 1 wherein identifying the cluster to sort is based on a user manually identifying the cluster.

6. The method of claim 1 wherein identifying the cluster to sort is based on machine learning to identify the cluster.

7. The method of claim 1 wherein fine-tuning the classification network includes performing training with an additional dataset based on the cluster.

8. An apparatus comprising:
a non-transitory memory for storing an application, the application for:
extracting one or more features from cell images using a neural network-based feature encoder;
clustering one or more cells from the cell images based on the extracted one or more features to generate one or more clusters;
identifying a cluster of the one or more clusters to sort;
fine-tuning a classification network based on the cluster; and
performing real-time live sorting of a set of cells using the classification network; and
a processor configured for processing the application.

9. The apparatus of claim 8 wherein the one or more features comprise a target protein based on a fluorescent dye.

10. The apparatus of claim 9 wherein clustering the one or more cells is based on a location of the target protein.

11. The apparatus of claim 10 wherein when the target protein is in the cytosol, the one or more cells are clustered as dormant cells, and when the target protein is in the nucleus, the one or more cells are clustered as activated cells.

12. The apparatus of claim 8 wherein identifying the cluster to sort is based on a user manually identifying the cluster.

13. The apparatus of claim 8 wherein identifying the cluster to sort is based on machine learning to identify the cluster.

14. The apparatus of claim 8 wherein fine-tuning the classification network includes performing training with an additional dataset based on the cluster.

15. A system comprising:
a first computing device configured for sending one or more cell images to the second computing device; and
a second computing device configured for:
extracting one or more features from the one or more cell images using a neural network-based feature encoder;
clustering one or more cells from the cell images based on the extracted one or more features to generate one or more clusters;
identifying a cluster of the one or more clusters to sort;
fine-tuning a classification network based on the cluster; and
performing real-time live sorting of a set of cells using the classification network.

16. The system of claim 15 wherein the one or more features comprise a target protein based on a fluorescent dye.

17. The system of claim 16 wherein clustering the one or more cells is based on a location of the target protein.

18. The system of claim 17 wherein when the target protein is in the cytosol, the one or more cells are clustered as dormant cells, and when the target protein is in the nucleus, the one or more cells are clustered as activated cells.

19. The system of claim 15 wherein identifying the cluster to sort is based on a user manually identifying the cluster.

20. The system of claim 15 wherein identifying the cluster to sort is based on machine learning to identify the cluster.

21. The system of claim 15 wherein fine-tuning the classification network includes performing training with an additional dataset based on the cluster.
